(19) United States
(12) Patent Application Publication
(10) Pub. No.: US 2023/0020692 A1
(43) Pub. Date: Jan. 19, 2023

(54) COMPOSITION FOR BRIGHTENING AND/OR WHITENING KERATIN MATERIALS

(71) Applicant: L'OREAL, Paris (FR)

(72) Inventors: Jie QIU, Shanghai (CN); Xiuxia WANG, Shanghai (CN); Marie-Jocelyne PYGMALION, Aulnay-sous-Bois (FR)

(73) Assignee: L'OREAL, Paris (FR)

(21) Appl. No.: 17/779,021

(22) PCT Filed: Dec. 9, 2019
(86) PCT No.: PCT/CN2019/124033

§ 371 Date: May 23, 2022

(51) Int. Cl. A61K 8/34; A61Q 19/02; A61K 8/9789; A61K 8/368

(52) U.S. Cl. CPC A61K 8/347; A61Q 19/02; A61K 8/9789; A61K 8/368; A61K 2800/28

(57) ABSTRACT

It relates to a composition for brightening and/or whitening keratin materials comprising: i) at least one flavonoid; ii) at least one hydroxylated diphenylmethane derivative of formula (I) below: (I) wherein: R₁ is selected from a hydrogen atom, a methyl group, a saturated or unsaturated, linear or branched hydrocarbon chain containing from 2 to 4 carbon atoms, an —OH group and a halogen, R₂ is selected from a hydrogen atom, a methyl group, and a saturated or unsaturated, linear or branched hydrocarbon chain containing from 2 to 5 carbon atoms, R₃ is selected from a methyl group or a saturated or unsaturated, linear or branched hydrocarbon chain containing from 2 to 5 carbon atoms, R₄ and R₅ are, independently of one another, selected from a hydrogen atom, a methyl group, a saturated or unsaturated, linear or branched hydrocarbon chain containing from 2 to 5 carbon atoms, an —OH group or a halogen; and iii) at least one skin peeling agent. It also relates to a cosmetic process for brightening and/or whitening keratin materials, in particular the human skin.

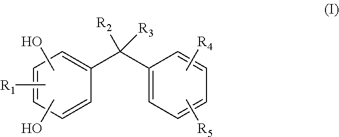

### TECHNICAL FIELD

The present invention relates to a cosmetic composition. In particular, the present invention relates to a composition for brightening and/or whitening keratin materials. The present invention also relates to a cosmetic process for brightening and/or whitening keratin materials.

### BACKGROUND ART

Human skin colour depends on various factors, and in particular on the seasons of the year, on race and on gender; it is mainly determined by the nature and the concentration of melanin produced by melanocytes. Melanocytes are specialized cells which synthesize melanin by means of specific organelles, melanosomes. In addition, at various times in their life, certain individuals experience the appearance of dark and/or coloured spots on the skin and more especially on the hands, which give the skin heterogeneity.

For various reasons associated in particular with greater comfort of use (softness, emollience and the like), current compositions for caring for and/or making up keratin materials, in particular the skin, are usually in the form of an emulsion of the oil-in-water (O/W) type consisting of an aqueous dispersing continuous phase and an oily dispersed discontinuous phase, or of an emulsion of the water-in-oil (W/O) type consisting of an oily dispersing continuous phase and an aqueous dispersed discontinuous phase.

O/W emulsions are the ones most sought in the cosmetics field, since they comprise an aqueous phase as the external phase, which gives them, when applied to the skin, a fresher, less greasy and lighter feel than W/O emulsions.

However, due to their natural feature, the conventional oil-in-water emulsions are not totally satisfying, in particular in terms of brightening and/or whitening of the skin.

Efforts have been made to introduce silicone resins, styrene type copolymers in combination with pigments and whitening active agents, into the oil-in-water emulsions. However the inventors found it difficult to obtain a composition with a good brightening and/or whitening effect on the skin.

Therefore, there exists a need for formulating a composition to provide a good brightening and/or whitening effect on keratin materials.

### SUMMARY OF THE INVENTION

The inventors have found that such a need can be achieved by the present invention.

Thus, according to one aspect, the present invention relates to a composition for brightening and/or whitening keratin materials comprising:

i) at least one flavonoid;

ii) at least one hydroxylated diphenylmethane derivative of formula (I) below:

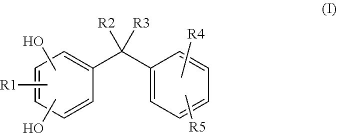

wherein:

R₁ is selected from a hydrogen atom, a methyl group, a saturated or unsaturated, linear or branched hydrocarbon chain containing from 2 to 4 carbon atoms, an —OH group and a halogen,

R₂ is selected from a hydrogen atom, a methyl group, and a saturated or unsaturated, linear or branched hydrocarbon chain containing from 2 to 5 carbon atoms,

R₃ is selected from a methyl group or a saturated or unsaturated, linear or branched hydrocarbon chain containing from 2 to 5 carbon atoms,

R₄ and R₅ are, independently of one another, selected from a hydrogen atom, a methyl group, a saturated or unsaturated, linear or branched hydrocarbon chain containing from 2 to 5 carbon atoms, an —OH group or a halogen; and

iii) at least one skin peeling agent.

The inventors found that the composition according to the present invention comprising a combination of ingredients i)-iii) above has a good brightening and/or whitening effect on keratin materials, in particular human skin.

According to another aspect, the present invention also relates to a cosmetic process for brightening and/or whitening keratin materials, in particular human skin, comprising the step of applying the composition according to the present invention on the keratin materials.

For the purposes of the present invention, the term “keratin material” is intended to cover human skin, mucous membranes such as the lips, the nails. Human skin, in particular facial skin, is most particularly considered according to the present invention.

Other subjects and characteristics, aspects and advantages of the present invention will emerge even more clearly on reading the detailed description and the examples that follow.

### BRIEF DESCRIPTION OF THE DRAWINGS

A more particular description of the present invention will be rendered by reference to the appended drawings, in which:

FIG. 1 shows the brightening and/or whitening effect of products A, B and C as well as the placebo prepared in Example 1;

FIG. 2 shows the brightening and/or whitening effect of product D prepared in Example 2 as well as the placebo prepared in Example 1; and

FIG. 3 shows the brightening and/or whitening effect of product E prepared in Example 3 as well as the placebo prepared in Example 1.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

According to one aspect, the present invention relates to a composition for brightening or whitening keratin materials comprising:

i) at least one flavonoid;

ii) at least one hydroxylated diphenylmethane derivative of formula (I) below:

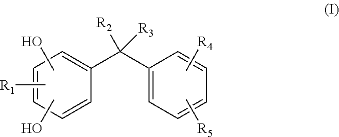

wherein:

R₁ is selected from a hydrogen atom, a methyl group, a saturated or unsaturated, linear or branched hydrocarbon chain containing from 2 to 4 carbon atoms, an —OH group and a halogen,

R₂ is selected from a hydrogen atom, a methyl group, and a saturated or unsaturated, linear or branched hydrocarbon chain containing from 2 to 5 carbon atoms,

R₃ is selected from a methyl group or a saturated or unsaturated, linear or branched hydrocarbon chain containing from 2 to 5 carbon atoms,

R₄ and R₅ are, independently of one another, selected from a hydrogen atom, a methyl group, a saturated or unsaturated, linear or branched hydrocarbon chain containing from 2 to 5 carbon atoms, an —OH group or a halogen; and

iii) at least one skin peeling agent.

Flavonoids

According to the first aspect, the composition according to the present invention comprises at least one flavonoid.

Flavonoids are a specific group of polyphenols, and are the most plentiful group of polyphenol compounds, making up about two-thirds of the total phenols in consumed feed. Flavonoids are further categorized, according to chemical structure, into chalcones, flavones, flavanones, flavanols, flavonols, dihydroflavonols, isoflavonoids, neoflavonoids, catechins, anthocyanidins, and tannins. Over 4,000 flavonoids have been identified, many of which occur in fruits, vegetables and beverages (tea, coffee, beer, wine and fruit drinks). The flavonoids have been reported to have antiviral, anti-allergic, antiplatelet, anti-inflammatory, antitumor and antioxidant activities. Flavonoids protect lipids and vital cell components from damaging oxidative stress by efficiently scavenging free radicals.

Preferably, the flavonoid used is flavone.

Baicalin, a component of Chinese medicinal herb Huang-chin, is a flavone, a type of flavonoid. It is a potent antioxidant that demonstrates potent effects against oxidative stress diseases, inflammation, allergy, cancer, bacterial infections, etc.

Baicalin is found in several species in the genus Scutellaria, including Scutellaria baicalensis and Scutellaria lateriflora. There are 10 mg/g baicalin in Scutellaria galericulata leaves. It is also present in the bark isolate of the Oroxylum indicum tree.

In one embodiment according to the present invention, baicalin is used in the form of scutellaria baiclensis root extract.

Advantageously, the flavonoid is present in an amount ranging from 0.01 wt. % to 2 wt. %, preferably from 0.05 wt. % to 1 wt. %, more preferably from 0.1 wt. % to 0.5 wt. %, relative to the total weight of the composition.

Hydroxylated Diphenylmethane Derivatives

According to the first aspect, the composition according to the present invention comprises at least one hydroxylated diphenylmethane derivative of formula (I) below:

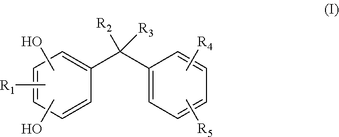

wherein:

R₁ is selected from a hydrogen atom, a methyl group, a saturated or unsaturated, linear or branched hydrocarbon chain containing from 2 to 4 carbon atoms, an —OH group and a halogen,

R₂ is selected from a hydrogen atom, a methyl group, and a saturated or unsaturated, linear or branched hydrocarbon chain containing from 2 to 5 carbon atoms,

R₃ is selected from a methyl group or a saturated or unsaturated, linear or branched hydrocarbon chain containing from 2 to 5 carbon atoms,

R₄ and R₅ are, independently of one another, selected from a hydrogen atom, a methyl group, a saturated or unsaturated, linear or branched hydrocarbon chain containing from 2 to 5 carbon atoms, an —OH group or a halogen.

The —OH, R₁, R₄ and R₅ groups may be in the ortho-, meta- or para-position with respect to the bond formed with the carbon linking the two aromatic nuclei to one another.

According to one preferred embodiment of the present invention, use is made of a compound of formula (I) wherein:
R₁, R₂, R₄ and R₅ denote a hydrogen atom;
R₃ is a methyl group;
the —OH groups are in the ortho- and para-position with respect to the bond formed with the carbon linking the two aromatic nuclei to one another.

This compound corresponds to formula (II) below:

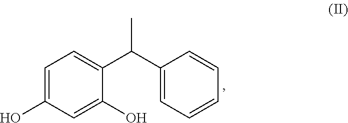 [SMILES: CC(c1ccccc1)c1ccc(O)cc1O]

known as 4-(1-phenylethyl)-1,3-benzenediol or 4-(1-phenylethyl)-1,3-dihydroxybenzene or otherwise known as phenylethyl resorcinol or phenylethylbenzenediol or styryl resorcinol. This compound has a CAS number 85-27-8. Such a compound is sold under the name Symwhite 377° or Bio 377 by the company Symrise.

Advantageously, the hydroxylated diphenylmethane derivative is present in amount ranging from 0.01 wt. % to 2 wt. %, more preferably from 0.05 wt. % to 1 wt. %, even more preferably from 0.1 wt. % to 0.5 wt. %, relative to the total weight of the composition.

Skin Peeling Agent

According to the first aspect, the composition according to the present invention comprises at least one skin peeling agent.

Without intending to be bound by theory, it is believed that skin peeling agent stimulates collagen production in the skin, thereby further enhancing the skin whitening effect.

According to a preferred embodiment according to the present invention, the skin peeling agent is selected from salicylic acid and derivatives thereof.

The salicylic acid derivative than can be used in the composition of the present invention is preferably selected from those of formula (III) below:

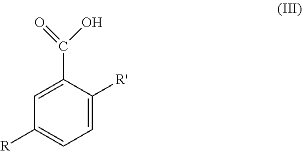

wherein:

R represents a hydrogen atom or a saturated or unsaturated, linear, branched or cyclic aliphatic alkoxy, ester or ketoxy chain, these chains comprising from 2 to 22 carbon atoms and possibly being substituted with at least one substituent selected from halogen atom, a trifluoromethyl group and hydroxyl groups in free form or esterified with an acid containing from 1 to 6 carbon atoms or else with a carboxyl function which is free or esterified with a lower alcohol containing from 1 to 6 carbon atoms; and

R′ represents a hydroxyl group or an ester function of formula (IV):

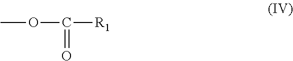

wherein R₁ is a saturated or unsaturated aliphatic group containing from 1 to 18 carbon atoms.

Preferably, the radical R comprises at least 4 carbon atoms. It is formed, for example, from a saturated linear alkyl or alkoxy radical containing from 4 to 11 carbon atoms.

Advantageously, the salicylic acid derivative is selected from salicylic acid, capryloylsalicylic acid, 5-n-decanoylsalicylic acid and 5-n-dodecanoyl-salicylic acid and is in particular capryloylsalicylic acid.

Advantageously, the peeling agent is present in an amount ranging from 0.01 wt. % to 2 wt. %, preferably from 0.05 wt. % to 1 wt. %, more preferably from 0.1 wt. % to 0.5 wt. %, relative to the total weight of the composition.

Aqueous Phase

The composition of the present invention comprises at least one continuous aqueous phase.

The aqueous phase of the composition according to the present invention comprises water and optionally one or more water-miscible or at least partially water-miscible compounds, for instance C₂ to C₈ lower polyols or monoalcohols, such as ethanol, isopropanol, and phenoxyethanol.

The term “polyol” should be understood as meaning any organic molecule comprising at least two free hydroxyl groups. Examples of polyols that may be mentioned include glycols, for instance butylene glycol, propylene glycol, and isoprene glycol, caprylyl glycol, glycerol (i.e. glycerin), ethylhexyl glycerin and polyethylene glycols.

The aqueous phase may also comprise any common water-soluble or water-dispersible additive as mentioned below.

The aqueous phase may represent from 30 wt. % to 98 wt. %, preferably from 30 wt. % to 95 wt. %, relative to the total weight of the composition.

Hydrotrope

According to some embodiments, the composition according to the present invention further comprises at least one hydrotrope.

A single type of hydrotrope may be used, but two or more different types of hydrotrope may be used in combination.

Hydrotropes (or hydrotropic agents) may be a diverse class of compounds characterized by an amphiphilic molecular structure and ability to dramatically increase the solubility of poorly soluble organic molecules in water. Many hydrotropes have aromatic structure with an ionic moiety, while some of them are linear alkyl chains, as listed in the table below. Although hydrotropes noticeably resemble surfactants and have the ability to reduce surface tension, their small hydrophobic units and relatively shorter alkyl chain distinguish them as a separate class of amphiphiles.

Common hydrotropic molecules include: sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N,N-diethylnicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid. Hydrotropes can be found in Lee J. et al., “Hydrotropic Solubilization of Paclitaxel: Analysis of Chemical Structures for Hydrotropic Property”, Pharmaceutical Research, Vol. 20, No. 7, 2003; and Hodgon T. K., Kaler E. W., “Hydrotropic Solutions”, Current Opinion in Colloid and Interface Science, 12, 121-128, 2007.

Cosmetically acceptable hydrotropes are preferable hydrotropes that can be used in cosmetic compositions. While hydrotropes represent a broad class of molecules used in various fields, cosmetic applications will be limited due to safety and tolerance restrictions. Preferred hydrotropes in cosmetics are listed as below:

| Name of hydrotropes | Structure |
|---|---|
| Nicotinamide (Vitamin B3) | 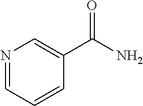 |
| Caffeine | 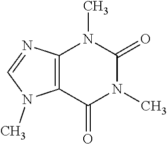 |
| Sodium PCA | 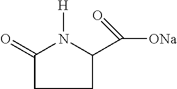 |
| Sodium Salicylate | 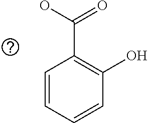 |

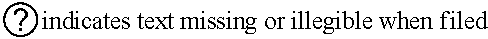

The suitability of a hydrotrope for use in cosmetic compositions can be determined using tests known in the art for determining effects of compounds on skin, and bioavailability methods.

An advantage of using hydrotropes is, once a stable solution is obtained, further dilution doesn't influence the stability of the solution. This is very different from organic solvents that are commonly used to increase the water solubility of actives. Typically, an aqueous dilution of organic solvents with pre-dissolved actives results in crystallization or precipitation.

The hydrotrope may have a log P being from −0.7 to 6, preferably from −0.7 to 1.0, preferably from −0.5 to 0.7 for non-ionic hydrotropes, and preferably from −0.7 to 5.5 for ionic hydrotropes (e.g. acidic hydrotropes).

Formulator will adjust pH in order to reach the best state of transparency with hydrotropes.

A log P value is a value for the base-ten logarithm of the apparent octan-1-ol/water partition coefficient. The log p values are known and are determined by a standard test which determines the concentration of the (c) compound in octan-1-ol and water. The log P may be calculated according to the method described in the article by Meylan and Howard: Atom/Fragment contribution method for estimating octanol-water partition coefficients, J. Pharm. Sci., 84: 83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

It is preferable that the hydrotrope be selected from oxothiazolidinecarboxylic acid, Vitamin B3 and derivatives thereof, preferably niacinamide, xanthine bases, preferably caffeine, camphor benzalkonium methosulfate, ellagic acid, hydroxyphenoxy propionic acid, diethyllutidinate, terephthalylidene dicamphor sulfonic acid, ferulic acid, phloretine, acetyl trifluoromethylphenyl valylglycine, resveratrol, 4-butylresorcinol, apigenin, phenylethyl resorcinol, prasterone, benzophenone-3, butylmethoxydibenzoylmethane, and jasmonic acid derivatives, preferably sodium tetrahydrojasmonate.

Vitamin B3 and derivatives thereof, xanthine bases such as caffeine, and jasmonic acid derivatives, which are described below in more detailed manner, are more preferable.

(Vitamin B3 and Derivatives Thereof)

Vitamin B3, also called vitamin PP, is a compound of the following formula (V):

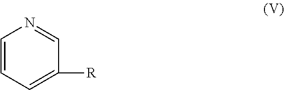

wherein R may be —CONH₂ (niacinamide), —COOH (nicotinic acid or niacin), or CH₂OH (nicotinyl alcohol), —CO—NH—CH₂—COOH (nicotinuric acid) or —CO—NH—OH (niconityl hydroxamic acid). Niacinamide is preferable.

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicyclic acid, glycolid acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

It is preferable that the Vitamin B3 or a derivative thereof has a log P being from −0.7 to 6, preferably from −0.6 to 5, more preferably −0.5 to 4.

(Xanthine Base)

Among the xanthine bases which may be used according to the present invention, mention may be made of: caffeine, theophylline, theobromine, acefylline, xanthinol nicotinate, diniprophylline, diprophylline, etamiphylline and its derivatives, etophylline, proxyphylline, pentophylline, propentophylline, pyridophylline, and bamiphylline, without this list being limiting.

It is preferable that the xanthine base be selected from the group consisting of caffeine, theophylline, theobromine, acefylline and mixtures thereof. These xanthine bases are known as inhibitors of phosphodiesterase, which is the enzyme responsible for the degradation of cAMP. By increasing the intracellular content of cAMP, these xanthine bases promote lipolytic activity and thus constitute first-rate slimming active agents.

As an example of plant extracts containing xanthine bases, mention may be made in particular of extracts of tea, of coffee, of guarana, of Paraguay tea, and of cola, without this list being limiting.

It is preferable that the xanthine base has a log P being from −0.7 to 6, preferably from −0.6 to 5, more preferably −0.5 to 4, and even more preferably from −0.3 to 2.

(Jasmonic Acid Derivative)

The jasmonic acid derivative is a compound selected from those corresponding to the following formula (VI):

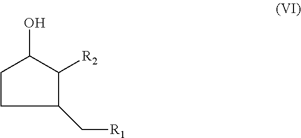

wherein:

R₁ represents a COOR₃ radical, R₃ denoting a hydrogen atom or a C₁-C₄ alkyl radical optionally substituted by one or more hydroxyl groups;

R₂ represents a hydrocarbon radical which is saturated or unsaturated, which is linear and which has from 1 to 18 carbon atoms or which is branched or cyclic and which has from 3 to 18 carbon atoms; and

their optical isomers, and corresponding salts.

Preferably, R₁ denotes a radical selected from —COOH, —COOMe (Me: methyl group), —COO—CH₂—CH₃, —COO—CH₂₋—CH(OH)—CH₂OH, —COOCH₂—CH₂—CH₂OH or —COOCH₂—CH(OH)—CH₃. Preferably R₁ denotes a —COOH radical.

Preferably, R₂ denotes a saturated or unsaturated linear hydrocarbon radical preferably having from 2 to carbon atoms. In particular, R₂ can be a pentyl, pentenyl, hexyl or heptyl radical.

According to one embodiment, the compound of formula (I) is selected from 3-hydroxy-2-[(2Z)-2-pentenyl]cyclopentaneacetic acid or 3-hydroxy-2-pentylcyclopentaneacetic acid and is preferably 3-hydroxy-2-pentylcyclopentaneacetic acid.

The salts of the compounds which can be used according to the present invention are chosen in particular from alkali metal salts, for example sodium or potassium salts; alkaline earth metal salts, for example calcium, magnesium or strontium salts; metal salts, for example zinc, aluminum, manganese or copper salts; salts of ammonium of formula NH₄⁺; quaternary ammonium salts; organic amine salts, such as, for example, methylamine, dimethylamine, trimethylamine, triethylamine, ethylamine, 2-hydroxyethylamine, bis(2-hydroxyethyl)amine or tris(2-hydroxyethyl)amine salts; or lysine or arginine salts. Use is preferably made of salts selected from sodium, potassium, calcium, magnesium, strontium, copper, manganese or zinc salts.

It is preferable to use the following compound as the jasmonic acid derivative (Mexoryl SBO).

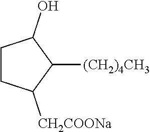

It is preferable that the jasmonic acid derivative has a log P being from −0.7 to 6, preferably from −0.6 to 5, more preferably −0.5 to 4.

If presents, the hydrotrope is present in an amount ranging from 0.1 wt. % to 10 wt. %, preferably from 0.5 wt. % to 5 wt. %, more preferably from 0.6 wt. % to 4 wt. %, relative to the total weight of the composition.

Gelling Agent

According to some embodiments, the composition according to the present invention further comprises at least one gelling agent.

Gelling agents can be used in the composition according to the present invention can be hydrophilic gelling agent and/or lipophilic gelling agent. Preferably, the composition according to the present invention comprises at least one hydrophilic gelling agent.

As preferred hydrophilic gelling agents, mention may be made more particularly of 2-acrylamido-2-methylpropanesulfonic acid polymers, for instance AMPS®, such as the ammonium 2-acrylamido-2-methylpropanesulfonate polymer sold under the trade name Hostacerin AMPS® by the company Clariant, and 2-acrylamido-2-methylpropanesulfonic acid copolymers and in particular copolymers of AMPS® and of hydroxyethyl acrylate, for instance the AMPS®/hydroxyethyl acrylate copolymer such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10° (INCI name: Hydroxyethyl acrylate/Sodium acryloyldimethyltaurate copolymer), copolymers of AMPS and of sodium acrylate, for instance AMPS/sodium acrylate copolymer such as the copolymer used in the commercial product sold under the name Simulgel™ EG by the company SEPPIC (CTFA name: ACRYLAMIDE/SODIUM ACRYLOYLDIMETHYLTAURATE COPOLYMER (and) ISOHEXADECANE (and) POLYSORBATE 80).

If presents, the gelling agent is present in the composition according to the present invention in an amount ranging from 0.1 wt. % to 5 wt. %, preferably from 0.3 wt. % to 3 wt. %, more preferably from 0.5 wt. % to 2 wt. % of the total weight of the composition.

Non-Ionic Surfactant

In some embodiments, the composition according to the present invention further comprises at least one non-ionic surfactant.

The non-ionic surfactant is selected from oxyalkylenated fatty acid esters of sorbitan, mono-esters of a polyol and a fatty acid containing from 8 to 24 carbon atoms, and mixtures of alkyl polyglycoside and fatty alcohol.

For oxyalkylenated fatty acid esters of sorbitan, mention may be made to:

oxyethylenated fatty acid esters of sorbitan such as (INCI name) Polysorbate 20, Polysorbate 40, Polysorbate 60, Polysorbate 65, Polysorbate 80, Polysorbate 85, PEG-5 sorbitan isostearate, PEG-20 sorbitan triisostearate, PEG-20 sorbitan isostearate, PEG-40 sorbitan septaoleate, PEG-20 sorbitan tetraoleate and PEG-20 sorbitan trioleate.

Mentions may be particulary made of polysorbate 80, such as those sold by the company Croda under the tradename TWEEN 80-LQ-(CQ).

For mono-esters of a polyol and a fatty acid containing from 8 to 24 carbon atoms, the polyol is preferably selected from glycerol, sorbitan, and diglycerol and the fatty acid is preferably selected from isolauric acid and oleic acid.

Examples of mono-esters of a polyol and a fatty acid containing from 8 to 24 carbon atoms include esters of polyglyceryl-2 laurate, glyceryl laurate, sorbitan oleate, and glyceryl oleate.

For mixtures of alkyl polyglycoside and fatty alcohol, mention can be made to a mixture of octyldodecyl xyloside and octyldodecanol, for example, that sold under the trade name Fluidanov 20 X® from the company SEPPIC, having the INCI name Octyldodecanol (and) Octyldodecyl Xyloside.

If presents, the non-ionic surfactant is present in an amount effective to ensure the stability of the composition according to the present invention.

If presents, the non-ionic surfactant is present in an amount ranging from 0.1 wt. % to 10 wt. %, relative to the total weight of the composition.

Oily Phase

In some embodiments, the composition of the present invention comprises at least one oily phase, dispersed in the aqueous phase as described above.

The nature of the oily phase of the composition according to the present invention is not critical.

In particular, the oily phase comprises at least one oil.

The term oil refers to any fatty substance in liquid form at room temperature (20-25° C.) and atmospheric pressure. These oils may be of animal, plant, mineral or synthetic origin.

The oils may be volatile or non-volatile.

The term “volatile oil” refers to any non-aqueous medium capable of evaporating from the skin or lips, in less than one hour, at room temperature (20-25° C.) and atmospheric pressure (760 mmHg). The volatile oil is a volatile cosmetic oil, liquid at room temperature. More specifically, a volatile oil has an evaporation rate of between 0.01 and 200 mg/cm²/min, inclusive.

The term “non-volatile oil” is intended to mean an oil remaining on keratin materials at ambient temperature and atmospheric pressure. More specifically, a non-volatile oil has an evaporation rate strictly below 0.01 mg/cm²/min.

To measure this evaporation rate, 15 g of oil or a mixture of oils to be tested are introduced into a crystallizer, 7 cm in diameter, placed on a scale located in a large 0.3 m³ chamber temperature-controlled at a temperature of 25° C., and humidity-controlled with a relative humidity of 50%. The liquid is left to evaporate freely, without stirring, by providing ventilation with a fan (PAPST-MOTOREN, reference 8550 N, rotating at 2700 rpm) positioned vertically above the crystallizer containing the solvent, with the blades directed toward the crystallizer and at a distance of 20 cm from the base of the crystallizer. The mass of oil remaining in the crystallizer is measured at regular intervals. The evaporation rates are expressed in mg of oil evaporated per surface area unit (cm²) and per time unit (minute).

The oil that is suitable for the present invention are not limited, it may be hydrocarbon-based, silicone-based or fluorine-based.

According to the present invention, the term “silicone oil” refers to an oil including at least one silicon atom, and in particular at least on Si—O group.

The term “fluorine oil” refers to an oil including at least one fluorine atom.

The term “hydrocarbon oil” refers to an oil containing primarily hydrogen and carbon atoms.

The oils may optionally include oxygen, nitrogen, sulfur and/or phosphorus atoms, for example, in the form of hydroxyl or acid radicals.

In an embodiment, isopropyl lauroyl sarcosinate and dimethicone are used as oil.

If presents, the oily phase is present in amount ranging from 1 wt. % to 50 wt. %, preferably from 3 wt. % to 40 wt. % and more preferably from 5 wt. % to 20 wt. %, relative to the total weight of the composition.

Additives

In a known manner, the composition of the present invention may also contain one or more additives that are common in cosmetics or dermatology.

Examples of adjuvants that may be mentioned include emulsifiers, other cosmetic active agents, preserving agents, antioxidants, fragrances, sunscreens (=UV-screening agents), basic agents (triethanolamine, diethanolamine or sodium hydroxide) or acidic agents (citric acid), vitamins (for example, tocopheryl acetate), and also lipid vesicles or any other type of vector (nanocapsules, microcapsules, etc.), and mixtures thereof.

These additives are used in the usual proportions in the cosmetics field, for example from 0.01% to 30% of the total weight of the composition.

These additives and the concentrations thereof must be such that they do not modify the desired properties for the composition of the present invention.

According to a preferred embodiment, the present invention relates to a composition for brightening or whitening keratin materials comprising, relative to the total weight of the composition:

i) from 0.1 wt. % to 0.5 wt. % of at least one flavone;

ii) from 0.1 wt. % to 0.5 wt. % of at least one hydroxylated diphenylmethane derivative selected from those of formula (I) below:

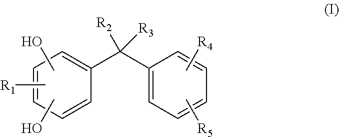

wherein:
R₁, R₂, R₄ and R₅ denote a hydrogen atom;
R₃ is a methyl group;
the —OH groups are in the ortho- and para-position with respect to the bond formed with the carbon linking the two aromatic nuclei to one another; and

iii) from 0.1 wt. % to 0.5 wt. % of at least one peeling agent selected from salicylic acid, capryloylsalicylic acid, 5-n-decanoylsalicylic acid and 5-n-dodecanoyl-salicylic acid.

According to another preferred embodiment, the present invention relates to a composition for brightening or whitening keratin materials comprising, relative to the total weight of the composition:

i) from 0.1 wt. % to 0.5 wt. % of at least one flavone;

ii) from 0.1 wt. % to 0.5 wt. % of at least one hydroxylated diphenylmethane derivative selected from those of formula (I) below:

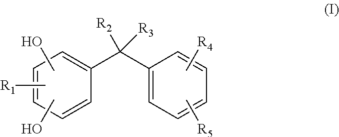

wherein:
R₁, R₂, R₄ and R₅ denote a hydrogen atom;
R₃ is a methyl group;
the —OH groups are in the ortho- and para-position with respect to the bond formed with the carbon linking the two aromatic nuclei to one another; and

(iii) from 0.1 wt. % to 0.5 wt. % of at least one peeling agent selected from salicylic acid, capryloylsalicylic acid, 5-n-decanoylsalicylic acid and 5-n-dodecanoyl-salicylic acid;

(iv) from 0.6 wt. % to 4 wt. % of at least one hydrotrope selected from oxothiazolidinecarboxylic acid, Vitamin B3 and derivatives thereof, preferably niacinamide, xanthine bases, preferably caffeine, camphor benzalkonium methosulfate, ellagic acid, hydroxyphenoxy propionic acid, diethyllutidinate, terephthalylidene dicamphor sulfonic acid, ferulic acid, phloretine, acetyl trifluoromethylphenyl valylglycine, resveratrol, 4-butylresorcinol, apigenin, phenylethyl resorcinol, prasterone, benzophenone-3, butyl methoxydibenzoylmethane, and jasmonic acid derivatives, preferably sodium tetrahydrojasmonate; and

(v) from 0.5 wt. % to 2 wt. % of at least one gelling agent.

The composition of the present invention is in the form of a lotion, cream, cosmetic water and serum and they are prepared according to the conventional methods in the cosmetic field.

Method and Use

The composition according to the present invention is intended for topical application and can especially constitute a composition intended for brightening and/or whitening keratin materials, and especially human skin.

Thus, in another aspect, the present invention relates to a cosmetic process for brightening and/or whitening keratin materials, in particular skin, comprising the step of applying the composition as defined above to the keratin materials.

The present invention is illustrated in greater detail by the examples described below, which are given as non-limiting illustrations.

The percentages are weight percentages by active ingredient, or active matters.

In the examples that follow, the weight percentages are indicated relative to the total weight of the composition.

### EXAMPLES

### Example 1

Products A, B and C and a placebo according to the formulas listed in the table below were prepared:

| | Placebo | Product A | Product B | Product C |
|---|---|---|---|---|
| INCI NAME | Wt. % | | | |
| PROPYLENE GLYCOL(1,2-PROPYLENEGLYCOL | 6 | 6 | 6 | 6 |
| from BASF) | | | | |
| PHENYL TRIMETHICONE(DOW CORNING 556 | 4 | 4 | 4 | 4 |
| COSMETIC GRADE FLUID from DOW CORNING) | | | | |
| PRUNUS ARMENIACA (APRICOT) KERNEL | 3 | 3 | 3 | 3 |
| OIL(REFINED APRICOT KERNEL OIL from OLVEA) | | | | |
| NYLON-12(TEGOLON 12-20 L from EVONIK | 5 | 5 | 5 | 5 |
| GOLDSCHMIDT) | | | | |
| PHENYLETHYL RESORCINOL(SYMWHITE ® 377 | — | 0.3 | 0.3 | — |
| from SYMRISE) | | | | |
| DIMETHICONE (and) DIMETHICONE/VINYL | 5 | 5 | 5 | 5 |
| DIMETHICONE CROSSPOLYMER(KSG 16 from | | | | |
| SHIN ETSU) | | | | |
| PEG/PPG-18/18 DIMETHICONE(DOW CORNING | 20 | 20 | 20 | 20 |
| 5225C FORMULATION AID from DOW CORNING) | | | | |
| GLYCERIN(GLYCERINE 4811 from OLEON) | 23 | 23 | 23 | 23 |
| SCUTELLARIA BAICALENSIS ROOT | — | — | 0.2 | 0.2 |
| EXTRACT(BAICALIN 95 MM from MMP)* | | | | |
| WATER | QS 100 | QS 100 | QS 100 | QS 100 |

*Note:
the amount of SCUTELLARIA BAICALENSIS ROOT EXTRACT was calculated by weight of baicalin.

The placebo does not comprise any of components i)-iii) according to the present invention and was used to determine a baseline in the study of effect of whitening effect.

Product A does not comprise components i) and iii) according to the present invention and is out of the present invention.

Product B does not comprise component iii) according to the present invention and is out of the present invention.

Product C does not comprise components ii) and iii) according to the present invention and is out of the present invention.

The products listed above were prepared according to a conventional process in the art.

Evaluation of Whitening Efficacy

The whitening efficacy of products A-C and the placebo were evaluated using a clinical protocol as follows.

The whitening efficacy of the different products was studied on the same subjects on delimited sites on the back. Products were applied at 4 mg/cm² on the delimited sites on the back, twice application a day. UV irradiation was carried out under solar simulator during 4 consecutive days. Colorimeter measurements were carried out with a chromameter Minolta CR400 (CIE L*a*b*).

ΔE: Total evolution of the color of the skin taking account of L*, a*, and b*. ΔE is calculated according to the following equation:

ΔE=\sqrt{Δa*²+Δb*²+ΔL*²}

The more this parameter is, the more the skin will be obscured.

The results of whitening efficacy of products A, B and C as well as the placebo were illustrated in FIG. 1.

It can be seen from FIG. 1 that product A didn't demonstrate any whitening effect, as compared with the placebo.

It can also be seen that products B and C demonstrated a whitening effect, as compared with the placebo, but not statistically significant.

### Example 2

Product D according to the formula listed in the table below was prepared with a conventional process in the art:

| INCI NAME | Wt. % |
|---|---|
| PROPYLENE GLYCOL(1,2-PROPYLENEGLYCOL | 6 |
| from BASF) | |
| NYLON-12(TEGOLON 12-20 L from EVONIK | 5 |
| GOLDSCHMIDT) | |
| PHENYL TRIMETHICONE(DOW CORNING 556 | 4 |
| COSMETIC GRADE FLUID from DOW CORNING) | |
| DIMETHICONE (and) DIMETHICONE/VINYL | 5 |
| DIMETHICONE CROSSPOLYMER | |
| PEG/PPG-18/18 DIMETHICONE(DOW CORNING | 20 |
| 5225C FORMULATION AID from DOW CORNING) | |
| PRUNUS ARMENIACA (APRICOT) KERNEL | 3 |
| OIL(REFINED APRICOT KERNEL OIL from OLVEA) | |
| GLYCERIN (GLYCERINE 4811 from OLEON) | 23 |
| PHENYLETHYL RESORCINOL(SYMWHITE ® 377 | 0.3 |
| from SYMRISE) | |
| CAPRYLOYL SALICYLIC ACID(MEXORYL SAB | 0.3 |
| from CHIMEX) | |
| WATER | QS 100 |

Product D does not comprise components i) according to the present invention and is out of the present invention.

The whitening efficacy of product D was evaluated using the clinical protocol as described in Example 1.

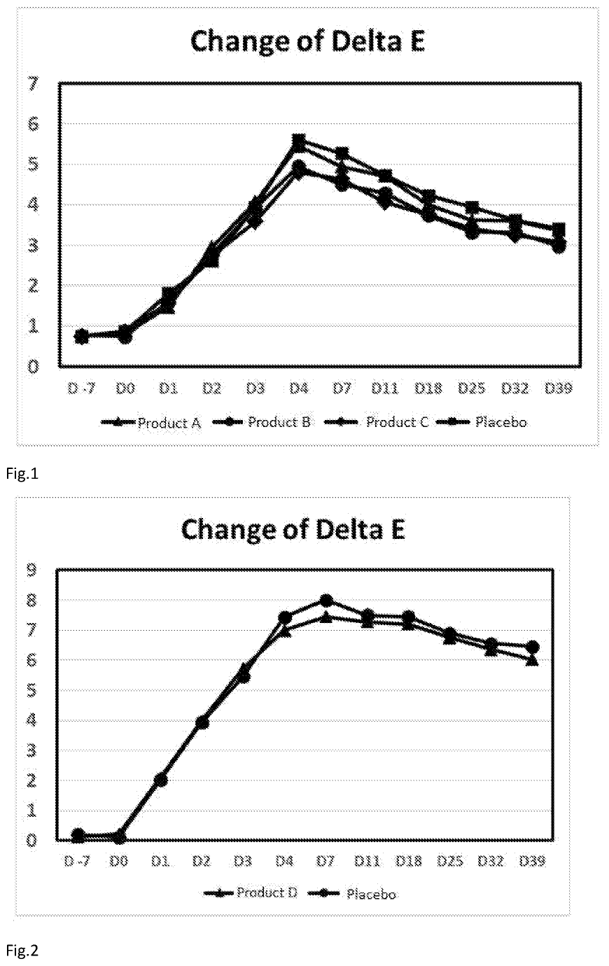

The results of whitening efficacy of product D as well as the placebo prepared in Example 1 were illustrated in FIG. 2.

It can be seen from FIG. 2 that product D didn't demonstrate any whitening effect, as compared with the placebo.

### Example 3

Product E according to the formula listed in the table below was prepared with a process as described below:

1. Preparation water phase: adding hydrotrope ingredient (NIACINAMIDE and/or CAFFEINE) into water and stirring till complete dissolution, then adding in SCUTELLARIA BAICALENSIS ROOT EXTRACT, stirring till complete dissolution, remaining the temperature at 75° C.

2. Preparation of oil phase: mixing PHENYLETHYL RESORCINOL, and oil together, heating to 75° C. to dissolve PHENYLETHYL RESORCINOL completely, then optionally adding POLYSORBATE 20.

3. Adding oil phase to water phase, with homogenization at 1000 rpm for 10 minutes by VMI type homogenising machine.

4. Cooling down to below 60° C., optionally adding HYDROXYETHYL ACRYLATE/SODIUM ACRYLOYLDIMETHYL TAURATE COPOLYMER and stirring until well dispersed and then add SODIUM HYDROXIDE to adjust pH to 5.0.

| INCI NAME | Product E |
|---|---|
| ISOPROPYL LAUROYL SARCOSINATE(ELDEW ® | 2 |
| SL-205 from AJINOMOTO) | |
| OCTYLDODECYL XYLOSIDE(FLUIDANOV ™ | 0.5 |
| 20 X from SEPPIC) | |
| PHENYLETHYL RESORCINOL(SYMWHITE ® | 0.3 |
| 377 from SYMRISE) | |
| ACRYLAMIDE/SODIUM ACRYLOYLDIMETHYLTAURATE | 1.08 |
| COPOLYMER(SIMULGEL ™ 600 from SEPPIC) | |
| OCTYLDODECANOL (FLUIDANOV ™ 20 X | 1.5 |
| from SEPPIC) | |
| GLYCERIN(GLYCERINE 4811 from OLEON) | 7 |
| CAFFEINE(CAFEINE ANHYDRE NATURELLE | 0.8 |
| POUDREfrom TESTA) | |
| DIMETHICONE(DOW CORNING TORAY SH200 C | 5.5 |
| FLUID 5CS from DOW CORNING) | |
| WATER | QS to 100 |
| CAPRYLOYL SALICYLIC ACID(MEXORYL SAB | 0.15 |
| from CHIMEX) | |
| POLYSORBATE 80(TWEEN 80-LQ-(CQ) from | 2 |
| CRODA) | |
| SCUTELLARIA BAICALENSIS ROOT EXTRACT(BAICALIN | 0.2 |
| 95 MM from MMP)* | |
| POLYSORBATE 20(TWEEN 20-LQ-(CQ) from CRODA) | 0.2 |

*Note:
the amount of SCUTELLARIA BAICALENSIS ROOT EXTRACT was calculated by weight of baicalin.

The whitening efficacy of product E was evaluated using the clinical protocol as described in Example 1.

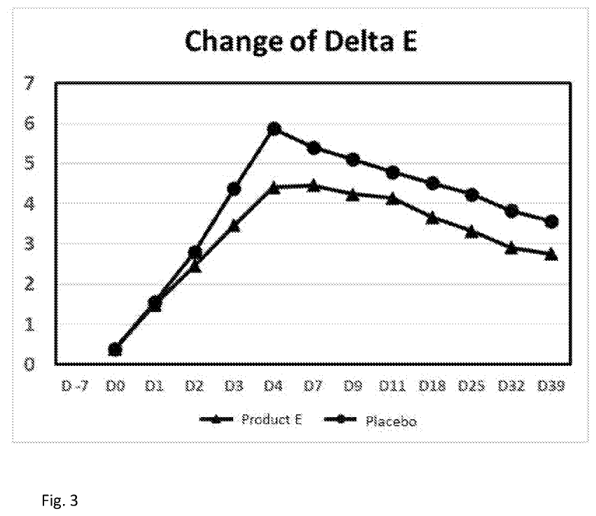

The results of whitening efficacy of product E as well as the placebo prepared in Example 1 were illustrated in FIG. 3.

It can be seen from FIG. 3 that product E demonstrated significant whitening effect, as compared with the placebo.

It was also found that product had good stability at room temperature for over 2 months.

### Example 4

A cream according to the present invention with the following formula was prepared with a similar preparation process as described in Example 3.

| INCI NAME | Wt. % |
|---|---|
| ISOPROPYL LAUROYL SARCOSINATE | 2 |
| (ELDEW ® SL-205 from AJINOMOTO) | |
| OCTYLDODECYL XYLOSIDE (FLUIDANOV ™ 20 X | 0.5 |
| from SEPPIC) | |
| BUTYROSPERMUM PARKII (SHEA) (FAIR FOR LIFE | 2 |
| REFINED SHEA BUTTER from OLVEA | |
| PHENYLETHYL RESORCINOL (SYMWHITE ® 377 from | 0.3 |
| SYMRISE) | |
| ACRYLAMIDE/SODIUM ACRYLOYLDIMETHYLTAURATE | 1.2 |
| COPOLYMER (SIMULGEL ™ 600 from SEPPIC) | |
| OCTYLDODECANOL (FLUIDANOV ™ 20 X | 1.5 |
| from SEPPIC) | |
| GLYCERIN (GLYCERINE 4811 from OLEON) | 7 |
| CAFFEINE (CAFEINE ANHYDRE NATURELLE POUDRE | 0.8 |
| from TESTA) | |
| DIMETHICONE(DOW CORNING TORAY SH200 C FLUID | 2 |
| 5CS from DOW CORNING) | |
| Water | QS to 100 |
| CAPRYLOYL SALICYLIC ACID(MEXORYL SAB from | 0.15 |
| CHIMEX) | |
| POLYSORBATE 80(TWEEN 80-LQ-(CQ) from CRODA) | 0.15 |
| DIMETHICONE(DOW CORNING TORAY SH200 C FLUID | 0.2 |
| 5CS from DOW CORNING) | |
| SCUTELLARIA BAICALENSIS ROOT EXTRACT (BAICALIN | 0.2 |
| 95 MM from MMP)* | |

*Note:
the amount of SCUTELLARIA BAICALENSIS ROOT EXTRACT was calculated by weight of baicalin.

According to the clinical protocol as described in Example 1, the cream obtained demonstrated significant whitening effect comparable to that of product E.

It was also found that the cream obtained had good stability at room temperature for over 2 months.

### Example 5

A cosmetic water according to the present invention with the following formula was prepared with a similar preparation process as described in Example 3.

| INCI NAME | Wt. % |
|---|---|
| ISOPROPYL LAUROYL SARCOSINATE(ELDEW ® | 0.5 |
| SL-205 from AJINOMOTO) | |
| OCTYLDODECYL XYLOSIDE(FLUIDANOV ™ 20 X | 0.13 |
| from SEPPIC) | |
| PHENYLETHYL RESORCINOL (SYMWHITE ® 377 | 0.3 |
| from SYMRISE) | |
| OCTYLDODECANOL (FLUIDANOV ™ 20 X from | 0.38 |
| SEPPIC) | |
| GLYCERIN (GLYCERINE 4811 from OLEON) | 3 |
| CAFFEINE (CAFEINE ANHYDRE NATURELLE POUDRE | 1 |
| from TESTA) | |
| WATER | QS to 100 |
| CAPRYLOYL SALICYLIC ACID(MEXORYL SAB from | 0.15 |
| CHIMEX) | |
| SCUTELLARIA BAICALENSIS ROOT EXTRACT (BAICALIN | 0.2 |
| 95 MM from MMP)* | |
| HYDROXYETHYL ACRYLATE/SODIUM ACRYLOYLDIMETHYL | 0.7 |
| TAURATE COPOLYMER(SEPINOV ™ EMT 10 from SEPPIC) | |
| DICAPRYLYL CARBONATE(CETIOL ® CC from BASF) | 0.5 |
| POLYSORBATE 80(TWEEN 80-LQ-(CQ) from CRODA) | 0.4 |

*Note:
the amount of SCUTELLARIA BAICALENSIS ROOT EXTRACT was calculated by weight of baicalin.

According to the clinical protocol as described in Example 1, the cosmetic water obtained demonstrated significant whitening effect comparable to that of product E.

It was also found that the cosmetic water obtained had good stability at room temperature for over 2 months.

### Example 6

A serum according to the present invention with the following formula was prepared with a similar preparation process as described in Example 3.

| INCI NAME | Wt. % |
|---|---|
| PHENYLETHYL RESORCINOL (SYMWHITE ® 377 from | 0.3 |
| SYMRISE) | |
| ACRYLAMIDE/SODIUM ACRYLOYLDIMETHYLTAURATE | 0.68 |
| COPOLYMER (SIMULGEL ™ 600 from SEPPIC) | |
| GLYCERIN (GLYCERINE 4811 from OLEON) | 6 |
| CAFFEINE (CAFEINE ANHYDRE NATURELLE POUDRE | 0.8 |
| from TESTA) | |
| WATER | QS to 100 |
| CAPRYLOYL SALICYLIC ACID(MEXORYL SAB from | 0.15 |
| CHIMEX) | |
| POLYSORBATE 80(TWEEN 80-LQ-(CQ) from CRODA) | 0.12 |
| SCUTELLARIA BAICALENSIS ROOT EXTRACT (BAICALIN | 0.2 |
| 95 MM from MMP)* | |
| POLYSILICONE-11(GRANSIL RPS-D6 from GRANT | 0.65 |
| INDUSTRIES) | |
| CAPRYLIC/CAPRIC TRIGLYCERIDE(DUB MCT 7030/MB | 3 |
| from STEARINERIE DUBOIS) | |
| BIS-PEG/PPG-16/16 PEG/PPG-16/16 DIMETHICONE(ABIL | 0.44 |
| CARE 85 from EVONIK GOLDSCHMIDT) | |
| PEG-20 METHYL GLUCOSE SESQUISTEARATE (GLUCAMATE | 0.2 |
| SSE-20 EMULSIFIER from LUBRIZOL) | |
| XANTHAN GUM (KELTROL CG-T from CP KELCO) | 0.2 |

*Note:
the amount of SCUTELLARIA BAICALENSIS ROOT EXTRACT was calculated by weight of baicalin.

According to the clinical protocol as described in Example 1, the serum obtained demonstrated significant whitening effect comparable to that of product E.

It was also found that the serum obtained had good stability at room temperature for over 2 months.

1. A composition for brightening and/or whitening keratin materials comprising:
i) at least one flavonoid;
ii) at least one hydroxylated diphenylmethane derivative of formula (I) below: 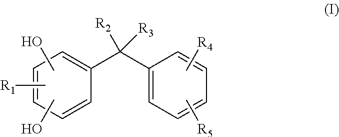
wherein:
R₁ is selected from a hydrogen atom, a methyl group, a saturated or unsaturated, linear or branched hydrocarbon chain containing from 2 to 4 carbon atoms, an —OH group and a halogen,
R₂ is selected from a hydrogen atom, a methyl group, and a saturated or unsaturated, linear or branched hydrocarbon chain containing from 2 to 5 carbon atoms,
R₃ is selected from a methyl group or a saturated or unsaturated, linear or branched hydrocarbon chain containing from 2 to 5 carbon atoms,
R₄ and R₅ are each independently, selected from a hydrogen atom, a methyl group, a saturated or unsaturated, linear or branched hydrocarbon chain containing from 2 to 5 carbon atoms, an —OH group or a halogen; and
iii) at least one skin peeling agent.

2. Composition according to claim 1, wherein the flavonoid is selected from the group consisting of chalcones, flavones, flavanones, flavanols, flavonols, dihydroflavonols, isoflavonoids, neoflavonoids, catechins, anthocyanidins, and tannins.

3. Composition according to claim 1, wherein the flavonoid is present in an amount ranging from 0.01 wt. % to 2 wt. % relative to the total weight of the composition.

4. Composition according to claim 1, wherein in formula (I):
R₁, R₂, R₄ and R₅ denote a hydrogen atom;
R₃ is a methyl group; and
the —OH groups are in the ortho- and para-position with respect to the bond formed with the carbon linking the two aromatic nuclei to one another.

5. Composition according claim 1, wherein the hydroxylated diphenylmethane derivative is present in amount ranging from 0.01 wt. % to 2 wt. % relative to the total weight of the composition.

6. Composition according claim 1, wherein the peeling agent is selected from salicylic acid and compounds of formula (III): 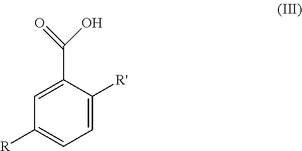
wherein:
R represents a hydrogen atom or a saturated or unsaturated, linear, branched or cyclic aliphatic alkoxy, ester or ketoxy chain, these chains comprising from 2 to 22 carbon atoms and optionally substituted with at least one substituent selected from halogen atom, a trifluoromethyl group and hydroxyl groups in free form or esterified with an acid containing from 1 to 6 carbon atoms or with a carboxyl function which is free or esterified with a lower alcohol containing from 1 to 6 carbon atoms; and
R′ represents a hydroxyl group or an ester function of formula (IV): 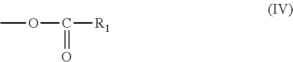
wherein R₁ is a saturated or unsaturated aliphatic group containing from 1 to 18 carbon atoms.

7. Composition according to claim 1, wherein the peeling agent is selected from the group consisting of salicylic acid, capryloylsalicylic acid, 5-n-decanoyisalicylic acid and 5-n-dodecanoyl-salicylic acid.

8. Composition according to claim 1, wherein the peeling agent is present in an amount ranging from 0.01 wt. % to 2 wt. % relative to the total weight of the composition.

9. Composition according to claim 1 further comprising at least one hydrotrope selected from the group consisting of oxothiazolidinecarboxylic acid, Vitamin B3 and derivatives thereof, xanthine bases, camphor benzalkonium methosulfate, ellagic acid, hydroxyphenoxy propionic acid, diethyllutidinate, terephthalylidene dicamphor sulfonic acid, ferulic acid, phloretine, acetyl trifluoromethylphenyl valylglycine, resveratrol, 4-butylresorcinol, apigenin, phenylethyl resorcinol, prasterone, benzophenone-3, butylmethoxydibenzoylmethane, and jasmonic acid derivatives.

10. Composition according to claim 9, wherein the hydrotrope is present in an amount ranging from 0.1 wt. % to 10 wt. % relative to the total weight of the composition.

11. Composition to according to claim 1 further comprising at least one gelling agent.

12. Composition according to claim 11, wherein the gelling agent is present in an amount ranging from 0.1 wt. % to 5 wt. % of the total weight of the composition.

13. Composition to according to claim 1 further at least one non-ionic surfactant selected from the group consisting of oxyalkylenated fatty acid ester of sorbitan, and mixtures of alkyl polyglycoside and fatty alcohol.

14. A composition for brightening or whitening keratin materials comprising, relative to the total weight of the composition:
i) from 0.1 wt. % to 0.5 wt. % of at least one flavone;
ii) from 0.1 wt. % to 0.5 wt. % of at least one hydroxylated diphenylmethane derivative selected from those of formula (I): 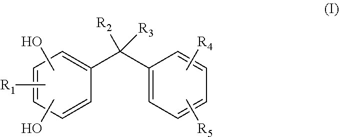
wherein:
R₁, R₂, R₄ and R₅ denote a hydrogen atom;
R₃ is a methyl group;
the —OH groups are in the ortho- and para-position with respect to the bond formed with the carbon linking the two aromatic nuclei to one another; and
iii) from 0.1 wt. % to 0.5 wt. % of at least one peeling agent selected from the group consisting of salicylic acid, capryloylsalicylic acid, 5-n-decanoylsalicylic acid and 5-n-dodecanoyl-salicylic acid.

15. A cosmetic process for brightening and/or whitening keratin materials, comprising applying the composition of claim 1 on the keratin materials.

16. Composition according to claim 1, wherein the peeling agent is capryloylsalicylic acid.
